(19) United States
(12) Patent Application Publication
(10) Pub. No.: US 2003/0158124 A1
(43) Pub. Date: Aug. 21, 2003

(54) Functionalized tannin and improved protein extractant

(75) Inventors: Jenn S. Shih, Paramus, NJ (US); Mustafa Rehmanji, Bloomingdale, NJ (US); Michael A. Drzewinski, Long Valley, NJ (US)

Correspondence Address: William J. Davis, Esq., IINTERNATIONAL SPECIALTY PRODUCTS, Building No. 10, 1361 Alps Road, Wayne, NJ, 07470, US

(73) Assignee: ISP INVESTMENTS INC.

(21) Appl. No.: 10/079,762

(22) Filed: Feb. 20, 2002

(51) Int. Cl. A61K 31/7024; A61K 31/325; C07C 269/02; C07C 69/84

(52) U.S. Cl. 514/23; 514/485; 536/53; 536/119; 560/104; 560/25

(57) ABSTRACT

A tannin functionalized with an aliphatically unsaturated, hydroxy reactive compound selected from the group of a carboxylic acid, a carboxylic acid ester, an isocyanate and an epoxide, which functionalized tannin is particularly useful in crosslinked form as an extractant in beverage clarification.

### FIELD OF THE INVENTION

This invention concerns a water soluble tannin functionalized with an aliphatically unsaturated compound which contains a hydroxy reactive carboxylic acid, carboxylic acid ester, isocyanate or glycidyl moiety as the functionalizing group and to the water insoluble crosslinked product of the functionalized tannin useful in the removal and/or recovery of proteins from liquid solutions, such as beer, wine, fruit juices and enzymic-containing organism solutions.

### BACKGROUND OF THE INVENTION

Many techniques have been developed to improve the flavor, appearance and shelf life of beverages. Chill haze or turbidity caused by certain proteins has been a major problem affecting shelf life; hence the removal of proteins, as well as phenolic compounds which are known to alter flavor and appearance, has been the subject of extensive research in the field of beverage clarification. Because of its known affinity for proteins, tannin has been proposed for treating beverages; however, the lengthy settling time and difficult titration required for tannin removal has limited its use. Also, residual tannins in beer adversely affect flavor. Alternatively, it has been proposed to immobilize tannin by adsorption on a support, e.g. silica, by the use of chemical bonding agents as reported in U.S. Pat. No. 4,500,554; however the immobilizing chemicals deleteriously affect food purity and introduce several other undesirable issues. Hence, there is a need for tannin-containing compounds or compositions that do not possess objectionable filtration problems or alter the taste or appearance of beverages.

Tannins in water-insoluble form on a carrier have also found application in selectively and reversibly adsorbing proteins for use as heterogeneous catalysts to induce enzymatic reactions. Enzymes and other biologically active proteins isolated from living organisms have been used as food additives, feed supplements, medicinals, reagents and industrial raw materials. Accordingly, tannin, in a non-altering-flavor form and non-reactive with other components in medicinal or food compositions, is highly desirable.

Accordingly, it is an object of this invention to address and overcome difficulties associated with the above applications by the use of a novel tannin compound which selectively adsorbs protein without interfering with other components in a liquid composition.

Another object is to provide an improved beverage clarifier and a more efficient clarification process.

Yet another object is the use of an improved tannin compound alone or in a blended formulation for clarification of beverages.

Still another object is to provide a commercially feasible and economical method for the preparation of a novel functionalized tannin and to the preparation of a crosslinked tannin as an improved protein adsorbent.

Yet another object is to provide a formulation including the functionalized tannin useful in many diverse applications.

These and other objects and benefits of the invention will become apparent from the following description and disclosure.

### SUMMARY OF THE INVENTION

This invention concerns (i) a tannin functionalized with an aliphatically unsaturated compound containing a carboxylic acid, carboxylic acid ester, isocyanate or epoxy moiety as a hydroxy reactive, functionalizing group and to the crosslinked product of the functionalized tannin; (ii) an economical method of forming the functionalized tannin and its crosslinked derivative and (iii) to the use of the crosslinked tannin compound as an improved protein absorbent, particularly effective in the clarification of beverages including beer, wine and fruit juices.

### DETAILED DESCRIPTION OF THE INVENTION

In accordance with this invention there is provided a novel functionalized tannin derived from the reaction between a tannin compound and a C₃ to C₁₂ compound having a terminal C₂ to C₃ alkylene unsaturated group and additionally containing a tannin functionalizing moiety selected from the group of a carboxylic acid, carboxylic acid ester, isocyanate and/or epoxy group which is reactive with a —OH group of the tannin to provide the functionalized tannin product while retaining a terminally unsaturated alkylene group required for subsequent crosslinking in the presence of an initiator.

The tannin component typically contains one or more compounds selected from the group of tannic acid, gallotannic acid, glucoside of tannic acid or glucoside of gallotannic acid, and the like which contain a plurality of reactive —OH sites.

In the following, the term (meth)acrylate is employed to include both acrylates and methacrylates. Similarly, (meth)acrylamide includes both acrylamides and methacrylamides.

The unsaturated functionalizing component generally contains a terminal vinyl or allyl group which remains intact after tannin functionalization and prior to crosslinking the functionalized product. Suitable examples of unsaturated functionalizing carboxylic acids and esters include acrylic, crotonic, itaconic, maleic, fumaric acids and their corresponding C₁ to C₄ esters and mixtures of these. Examples of functionalizing isocyanates include 2-isocyanato ethyl (meth)acrylate, 2-isocyanato (meth)acrylamide and the like. Representative unsaturated epoxy functionalizing agents include gylcidyl (meth)acrylate, lower alkyl substituted glycidyl (meth)acrylates, a C₆ to C₂₀ allyl glycidyl ether, a C₅ to C₂₀ vinyl glycidyl ether and mixtures thereof and mixtures with any of the foregoing functionalizing agents. Of this group, glycidyl methacrylate (GMA) is most preferred. The reaction employing glycidyl methacrylate is described as:

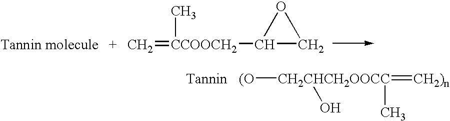

The reaction employing acrylic acid can be described as:

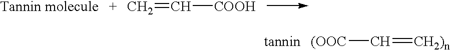

Similarly, when isocyanatoethyl methacrylate is the coreactant, the reaction is:

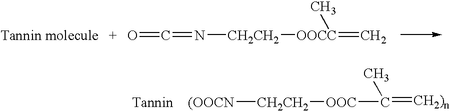

In the foregoing reactions, one or more of the —OH groups, preferably at least a plurality of the —OH groups, in the tannin molecule are reacted with the functionalizing coreactant.

The present functionalized tannin monomer is formed by introducing the tannin compound into an alkaline water solution having a pH of between about 7.5 and about 14. After the addition of tannin and before the addition of functionalizing agent, the reaction solution becomes noticeably more acidic. Between about 1.5 and about 180 wt. %, preferably between about 35 and about 130 wt. % functionalizing agent, based on tannin, is then added to the aqueous solution. Most desirably a concentration of 60-100% functionalizing agent with respect to tannin produces the desired monomer. The functionalizing reaction is effected in solution at a temperature of from about 20° C. to 150° C. with constant agitation over a period of from about 1 to 50 hours, preferably 5-10 hours. More often a temperature of between 40° C. and 100° C. is sufficient to produce the functionalized tannin monomeric compound and a reaction temperature of 50-80° C. with glycidyl methacrylate is particularly recommended.

The base initially used to provide the aqueous reaction solution, includes the hydroxides of Ca, K, Na, Li and the like and the wt. % of hydroxide is maintained at a dry basis concentration of between about 2 and about 50%, more desirably between 5 and 25%, based on the weight of tannin. Between 10 and 20% NaOH or KOH per weight of tannin is particularly desirable.

Optionally, up to 50 wt. % of vinyl pyrrolidone (VP), phenol absorber, can be added to the functionalized monomer and polymerized therewith to provide a mixed absorbent formulation. This allows the VP to be polymerized along with the functionalized tannin and may be employed in certain cases where it is desired to reduce the amount of tannin component in the formulation. However, the addition of VP is not needed for complete removal of phenols and protein from beverages.

After the tannin functionalization reaction is complete, the resulting monomer is polymerized and crosslinked by actinic radiation, e.g. UV-radiation in the presence or absence of a photoinitiator, e.g. Darocur® 1173, Irgacure® 2959 etc., or thermally by the addition of a water soluble, high temperature, free radical initiator, e.g. a peroxy initiator such as LUPERSOL® 11, 101, 80, dimethylazodiiso butyrate (WAKO) and azobis nitrile initiators such as VAZO 52, 64, 67 and 88 or a mixture thereof, the perester initiators, such as tert-butyl peroxy pivalate (Lupersol 11) being preferred. The resulting crosslinked product precipitates out of solution and any residual unreacted monomer contained in the precipitate is removed by extraction with water or an aqueous alcoholic solution. The crosslinked product is then recovered and reduced to a desired particulate size by drying and milling using methods well known by those skilled in the art. As an alternative to adding VP to the functionalized tannin prior to polymerization, a minor amount of crosslinked poly(vinylpyrrolidone), e.g. POLYCLAR, can be added to the crosslinked tannin component to regulate the amount of protein removal and to provide a mixed formulation for the removal of contaminants.

The resulting crosslinked polymer in particulate form can be added to a protein-containing liquid for absorption and removal of protein and/or protein/phenol components. The amount of crosslinked, functionalized tannin used to treat the protein-containing liquid is preferably between about 1-1.2:1 moles based on the desired moles of protein to be removed. Although larger amounts of crosslinked, functionalized tannin can be used, no advantage is achieved. The amount of protein removed from a beverage can be regulated by the duration of contact and/or the concentration of the crosslinked tannin component in the treatment medium.

In another aspect of the invention, the above monomeric solution of functionalized tannin can be coated on an inorganic carrier, such as alumina, silica, clay etc. and then polymerized and crosslinked on the carrier surface before contacting with the protein containing liquid. This later procedure has the advantage of providing increased surface area for the active component and in cases where the carrier is not completely coated with the crosslinked, functionalized tannin, the former may also serve to absorb some protein or phenolic materials.

Having generally described the invention, reference is now had to the following examples which illustrate preferred embodiments of the discovery herein disclosed but which are not to be construed as limiting to the scope of this invention.

### EXAMPLE 1

### Synthesis of Tannin-GMA Pre-Polymer

Into a 500 ml 4-necked resin kettle, fitted with half-moon Teflon blade agitator, a nitrogen purge adapter, and a reflux condenser, charge 12 g of Tannin and 2.3 g of sodium hydroxide and 50 g of D.I. water (pH=9.5). The mixture was mixed at 200 rpm throughout the process. After the tannin was completely dissolved, 5.325 g of glycidyl methacrylate was added and stirred for 4-5 days at room temperature whereupon the tannin-GMA prepolymer is recovered as a water soluble liquid.

### EXAMPLE2

### Synthesis of Tannin-GMA Pre-Polymer

Into a 500 ml 4-necked resin kettle, fitted with half-moon Teflon blade agitator, a nitrogen purge adapter, and a reflux condenser, was charged 12 g of Tannin and 0.6 g of sodium hydroxide and 70 g of D.I. water. The mixture was stirred at 200 rpm throughout the process. After tannin was completely dissolved, 5.38 g of glycidyl methacrylate was added, the solution was heated to 50° C. and stirred for 5 hours at room temperature whereupon the tannin-GMA water soluble liquid was formed.

### EXAMPLE 3

### Synthesis of Tannin-GMA Pre-Polymer

Into a 1-liter, 4-necked resin kettle, fitted with an anchor agitator, a thermocouple, and a reflux condenser, was charged 200 g of Brewtan and 600 g of D.I. water. The agitator, operating at 200 rpm was turned on and the mixture was mixed throughout the process. A clear solution was obtained, after which 5.04 g of sodium hydroxide and 54 g of glycidyl methacrylate were charged and the resulting solution was stirred at room temperature overnight. The water soluble prepolymer product of the reaction was recovered.

### EXAMPLE 4

### Tannin-GMA Crosslinked Polymer

The solution of Example 3 was heated to 60° C. and 5 g of Lupersol 11 was charged under continuous mixing and purging with nitrogen. The resulting crosslinked polymer began to precipitate out of solution. Mixing was continued at 70° C. for an additional 5 hours, after which the precipitated polymer was separated by filtration and washed with water until a colorless filtrate of about 7 pH was obtained. The precipitate was then transferred to a blender wherein it was reduced to an average particle size of from about 50 to 100 μm. The precipitated polymer particles were then dried in a vacuum oven at 90° C.

### EXAMPLE 5

The crosslinked tannin product of Example 4 (5 g) was added to 1 liter of unstabilized beer at room temperature over a period of 24 hours with continuous shaking. The resulting mixture was then filtered through a 1.6 μm syringe filter and the treated beer supernate tested for protein content by the PT-STANDARD Test using a nephelometric titrator; the higher the titration value, the greater the degree of protein removal. The results are expressed as ml extractant/100 ml of beer and as reported in Table A (Sample 1).

### EXAMPLE 6

Example 5 was repeated except after filtration through a 1.6 μm syringe filter, saturated ammonium sulfate solution (SASS) was titrated into the beer while simultaneously measuring haze. The limit of precipitation is expressed as the volume of SASS at the beginning of the appearance of haze. The higher the titration value, the greater the degree of protein removal. The results of this test are also reported in Table A (Sample 2.

TABLE A

TITRATION VALUE

| SAMPLE | of untreated beer | of treated beer |
|---|---|---|
| 1 | 10.41 | 36.32 |
| 2 | 13.6 | 18.9 |

What is claimed is:

1. A tannin functionalized with an aliphatically unsaturated C₂ to C₁₂, hydroxy-reactive compound containing the functionalizing moiety selected from the group consisting of a carboxylic acid, carboxylic acid ester, isocyanate and a glycidyl moiety and a mixture thereof.

2. The functionalized tannin of claim 1 wherein the functionalizing moiety is a glycidyl moiety.

3. The functionalized tannin of claim 1 wherein the functionalizing moiety is an allyl glycidyl moiety.

4. The functionalized tannin of claim 1 wherein said tannin is selected from the group consisting of tannin acid, gallotannin acid, the glucoside of tannic acid and mixtures thereof.

5. The functionalized tannin of claim 1 wherein said compound is glycidyl methacrylate and the tannin is tannic acid; said functionalized tannin contains the group: 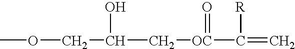
wherein R is hydrogen or methyl at one or more of the —OH sites on the tannic acid.

6. The functionalized tannin compound of one of claims 1, 2, 3, 4 or 5 in which the functionalized tannin is crosslinked at a site of said aliphatic unsaturation.

7. A tannin compound functionalized with a glycidyl coupound containing a terminal C₂ to C₃ alkylene group; which functionalization occurs as a result of epoxy ring opening and bonding to a hydroxy group of the tannin compound.

8. A tannin compound functionalized with a carboxylic acid containing an aliphatically unsaturated C₂ to C₃ group which functionalization occurs as a result of the reaction between the carboxylic group and a hydroxy group of the tannin.

9. A tannin compound functionalized with a carboxylic lower alkyl ester containing an aliphatically unsaturated C₂ to C₃ group which functionalization occurs as a result of the reaction between the ester group and a hydroxy group of the tannin.

10. A tannin compound functionalized with an isocyanate compound containing an aliphatically unsaturated C₂ to C₃ group which functionalization occurs as a result of the reaction between the isocyanate group and a hydroxy group of the tannin.

11. The crosslinked product of one of claims 7, 8, 9 or 10 which product is the result of crosslinking at the aliphatically unsaturated sites of the functionalized tannin.

12. The method which comprises contacting a beverage with an effective protein absorbing amount of the crosslinked product of one of claims 7, 8, 9 or 10.

13. The process of producing the crosslinked product of claim 11 which comprises:
(a) introducing a tannin selected from the group consisting of tannic acid, gallotannin acid, a glucoside of tannic acid and a glucoside of gallotannic acid into an aqueous solution having a pH between 7.5 and 14;
(b) reacting the tannin with between about 1.5 and about 180 wt. %, based on tannin, of a terminally substituted C₂ to C₃ aliphatically unsaturated compound containing a hydroxy-reactive group selected from the group consisting of a carboxylic acid, a carboxylic ester, isocyanate and epoxy at a temperature of between about 20° and about 150° C. and agitating until a uniform mixture is obtained;
(c) crosslinking the resulting functionalized tannin by exposure to actinic light or in the presence of a free radical initiator;
(d) precipitating the crosslinked product and washing with water to remove any unreacted monomer;
(e) drying the product and milling to a desired particle size.

14. The process of claim 13 wherein a minor amount of vinyl pyrrolidone monomer is mixed with the functionalized tannin, before crosslinking.

15. The process of claim 13 wherein a minor amount of crosslinked poly(vinyl pyrrolidone is added to the crosslinked, functionalized tannin product before milling.

16. The process of claim 13 wherein the functionalized tannin is coated on an inert inorganic carrier and is crosslinked on the surface of the carrier.
